(19) United States
(12) Patent Application Publication
(10) Pub. No.: US 2021/0166812 A1
(43) Pub. Date: Jun. 3, 2021

(54) APPARATUS AND METHODS FOR THE MANAGEMENT OF PATIENTS IN A MEDICAL SETTING

(71) Applicant: DIAGNOSTIC ROBOTICS LTD., Tel Aviv (IL)

(72) Inventors: Yonatan Amir, Jerusalem (IL); Moshe Shoham, Hoshaya (IL); Kira Radinsky, Zichron Yaakov (IL)

(21) Appl. No.: 16/616,610

(22) PCT Filed: May 31, 2018
(86) PCT No.: PCT/IB2018/053869

§ 371 Date: Nov. 25, 2019

Related U.S. Application Data
(60) Provisional application No. 62/514,023, filed on Jun. 2, 2017.

(51) Int. Cl. G16H 50/20; G06F 3/01; G16H 10/40; A61B 5/28; A61B 5/318

(52) U.S. Cl. CPC G16H 50/20; G06F 3/015; A61B 5/318; A61B 5/28; G16H 10/40

(57) ABSTRACT

Apparatus and methods are described for performing a differential diagnosis of a patient, including, using a computer processor (18), in a machine-learning stage, receiving data relating to a plurality of patients, receiving conditions that respective patients are diagnosed as having, and thereby determining correlations between respective patient parameters and the conditions that patients are diagnosed as having. In a patient-diagnosis stage, one or more conditions that a given patient is suspected of having are determined. Based at least partially upon the correlations determined during the machine-learning stage and the conditions that the given patient is suspected of having, a set of questions to ask the patient is determined such that determining that the patient has one of the conditions with a likelihood that passes a threshold likelihood may be achieved with a minimum number of questions being asked. Other applications are also described.

### CROSS-REFERENCE TO RELATED APPLICATIONS

The present application claims priority from US Provisional Patent Application No. 62/514,023 to Amir, filed Jun. 02, 2017, entitled “System for the management of patients in a medical setting,” which is incorporated herein by reference.

### FIELD OF EMBODIMENTS OF THE INVENTION

The present invention relates to methods and apparatus for use in emergency rooms and in other clinical settings, and particularly apparatus and methods that are used to increase the diagnostic accuracy and the patient throughput during emergency room or clinical procedures.

### BACKGROUND

The execution of emergency room medicine is usually described as being made up of five elements, which are as follows:

(i) identification;

(ii) triage;

(iii) anamnesis;

(iv) diagnosis; and

(v) prognosis.

Emergency room medicine is characterized by two distinctive features, which makes emergency room practice dissimilar from that of other hospital functional units. The first feature is the need for speedy handling and diagnosis of the patients. This requirement is compounded by the need to operate in situations which are often high load situations, especially in situations of mass casualty events, but also in extreme weather conditions, when elderly and weak patients are highly prone to illnesses. The second feature follows partly from the need for speedy diagnoses, and relates to the accuracy of the diagnosis and recommended treatment that can be achieved, under the conditions of urgency and load in a typical emergency room setting. The accuracy of the diagnosis can have a bearing on decisions regarding priority of treatment, which could have repercussions on the survival rate both of the patient being examined, and of other patients. Speedy diagnosis is especially important for medical conditions relating to cerebrovascular incidents, strokes and cardiac events. Furthermore, statistics have been presented indicating that incorrect diagnosis is a common cause of death in the medical field, and that 30 percent of such events occur as a result of a delay in treatment or incorrect diagnosis in the setting of the emergency room.

### SUMMARY OF EMBODIMENTS

In accordance with some applications of the present invention, at least one computer processor is configured to assess a patient's clinical condition within a medical setting in an autonomous or semi-autonomous manner. Typically, the at least one computer processor includes a computer processor of a patient-testing station, and the computer processor combines multiple sources of medical information, as obtained, inter alia, from:

(i) tests and examinations performed using the patient-testing station, a robotic station of the patient-testing station, and/or robotic components of the patient-testing station, e.g., using non-contact, contact and minimally invasive sensors,

(ii) machine interpretation and processing of medical images generated during the tests,

(iii) information obtained automatically and interactively at a patient-testing station, and

(iv) the patient's medical history,

together with artificial intelligence interrogation of databases of medical situations for comparing with and analyzing the above assembled patient medical information.

Typically the computer processor connects to historical databases of previous measurements (e.g., textual, structural, and/or visual measurements, etc.), and uses machine-learning based methods and/or general artificial-intelligence methods to mine patterns with high correlation to previously given patient anamnesis, diagnosis, prognosis, etc. Using the derived patterns, the computer processor typically applies such patterns to the analysis of current patients to classify them based on the historical patterns to the most probable diagnosis, prognosis, etc.

For some applications, a machine-learning stage is performed during which the at least one computer processor receives data relating to a plurality of patients, as well as conditions that respective patients belonging to the plurality of patients are diagnosed as having. By analyzing the aforementioned inputs, the computer processor determines correlations between respective patient parameters and the conditions that the patients are diagnosed as having. By way of example, the computer processor may determine that, whether or not a patient feels chest pains, is highly correlated with diagnosing a patient as suffering from a cardiac arrest, or it may determine that the age and/or sex of a patient is highly correlated with their susceptibility to liver disease.

During a patient diagnosis stage, a given patient is typically assessed. For some applications, the computer processor determines that the patient is suspected as having one or more conditions. For example, the computer processor may determine the one or more conditions that the patient is suspected of having, by asking the patient a preliminary set of questions, by automatically measuring one or more physiological parameters of the given patient using one or more sensors and/or one or more imaging devices, by automatically measuring one or more physiological parameters of the given patient using one or more robotic components, and/or by accessing the patient's medical history.

Subsequently, based at least partially upon the correlations determined during the machine-learning step, and based at least partially upon the one or more conditions that the given patient is suspected of having, the computer processor determines a set of questions to ask the patient. Typically, the set of questions is determined by selecting a set of questions that is such as to (a) determine that the patient has one of the one or more conditions with a likelihood that passes a threshold likelihood, with (b) a minimum number of questions being asked.

For some applications, the computer processor then chooses which question to ask the patient next, based upon the output of the previous step.

Typically, the machine-learning stage is ongoing and temporally overlaps with the patient-diagnosis stage, inasmuch that at the same time as a given patient is diagnosed, data relating to that patient is fed into the one or more computer processors that are configured to perform the machine-learning analysis of the data. For some applications, the data received in the machine-learning stage is received automatically, e.g., by being received from the computer processors of patient-testing stations, and/or by being received from other sources, e.g., by analyzing the medical records of a large number of patients that are stored on a database.

For some applications, during the machine-learning stage, the computer processor generates predictive models that relate patient-related data to patient diagnoses, for example, using machine-learning techniques. During the patient-diagnosis phase, the computer processor receives parameters relating to a given patient. For example, such parameters may be obtained by asking the patient questions via a user interface, by automatically measuring one or more physiological parameters of the given patient using one or more sensors and/or imaging devices, by automatically measuring one or more physiological parameters of the given patient using one or more robotic components, and/or by accessing the patient's medical history. In response to the received parameters, and using the predictive models that were determined during the machine-learning step, the computer processor diagnoses the patient as having one or more conditions.

For some applications, the computer processor generates an output indicating the one or more conditions that the patient is suspected of having, and additionally generates an output indicating the contribution of a given portion of the parameters, toward diagnosing the patient as having the one or more conditions. For example, the computer processor may generate an output indicating that the main contributing factor toward diagnosing the patient as having a given condition was his/her answer to a given question, was the result of a given test that was performed, was the result of an image that was acquired, was the result of an item in his/her medical history, etc. Alternatively or additionally, the computer processor may indicate a weighting of a given one of the parameters (or respective weightings of a plurality of parameters) in diagnosing the patient as having the given condition. For some applications, the computer processor generates a textual explanation of how the diagnosis was arrived at. For example, the text may include a description of a correlation between a given set of the received parameters and a condition that the patient has been diagnosed as having.

Typically, by outputting the indication of the contribution of a given portion of the parameters toward diagnosing the patient as having the one or more conditions, the confidence of the patient, and moreover, the confidence of the doctor in the machine-generated diagnosis is strengthened. Alternatively, by outputting the indication of the contribution of a given portion of the parameters toward diagnosing the patient as having the one or more conditions, the doctor is able to better assess whether he/she agrees with the diagnosis, and/or whether he/she would like any additional tests or examinations to be performed.

There is therefore provided, in accordance with some applications of the present invention, apparatus for performing a differential diagnosis of a patient, the apparatus including:

an output device; and

at least one computer processor configured:
in a machine-learning stage:
   to receive data relating to a plurality of patients, and to receive conditions that respective patients belonging to the plurality of patients are diagnosed as having; and
   to thereby determine correlations between respective patient parameters and the conditions that patients are diagnosed as having; and in a patient-diagnosis stage:
   to determine one or more conditions that a given patient is suspected of having;
   based at least partially upon the correlations determined during the machine-learning stage, and based at least partially upon the one or more conditions that the given patient is suspected of having, to determine a set of questions to ask the patient such that determining that the patient has one of the one or more conditions with a likelihood that passes a threshold likelihood may be achieved with a minimum number of questions being asked;
   based upon the determined set of questions, to choose a next question to ask the given patient; and
   to output the next question to the given patient, via the output device.

In some applications the computer processor is configured to choose the next question to ask the given patient by choosing a question the answer to which would carry the greatest weight in diagnosing the patient as suffering from the one of the one or more conditions with more than the threshold likelihood.

In some applications, the computer processor is further configured, in response to receiving a response from the given patient to one of the set of questions that indicates that it is more likely that the patient is suffering from a different condition from the one of the one or more conditions, to determine a new set of questions to ask the subject.

In some applications, the computer processor is configured to determine the set of questions that the given patient should be asked by using natural language processing to determine which words to use in the set of questions.

In some applications, the computer processor:

is further configured, in the machine-learning-stage, to identify a question that can be asked to a patient in order to resolve a contradiction between responses that the patient has given to two or more previous questions, and

is configured, in the patient-diagnosis stage, to choose the next question to ask the given patient by choosing to ask the given patient the identified question, in response to the given patient having given responses to two or more previous questions that result in the contradiction.

In some applications, the computer processor is configured to determine the one or more conditions that the given patient is suspected of having, at least partially by:

asking the given patient a preliminary set of questions; and

determining the one or more conditions that the given patient is suspected of having, based upon the responses of the given patient provides to the preliminary set of questions.

In some applications, the computer processor is configured to determine the one or more conditions that the given patient is suspected of having, at least partially by:

automatically measuring one or more physiological parameters of the given patient, using one or more sensors; and

determining the one or more conditions that the given patient is suspected of having, based upon the one or more physiological parameters.

In some applications, the computer processor is configured to determine the one or more conditions that the given patient is suspected of having, at least partially by:

automatically measuring one or more physiological parameters of the given patient, using one or more robotic components; and

determining the one or more conditions that the given patient is suspected of having, based upon the one or more physiological parameters.

In some applications, the computer processor is configured to determine the one or more conditions that the given patient is suspected of having, at least partially by:

accessing the patient's medical history; and

determining the one or more conditions that the given patient is suspected of having, based upon the patient's medical history.

There is further provided, in accordance with some applications of the present invention, a method for performing a differential diagnosis of a patient, the method including:

using at least one computer processor:
in a machine-learning stage:
   receiving data relating to a plurality of patients, and receiving conditions that respective patients belonging to the plurality of patients are diagnosed as having; and
   thereby determining correlations between respective patient parameters and the conditions that patients are diagnosed as having; and in a patient-diagnosis stage:
   determining one or more conditions that a given patient is suspected of having;
   based at least partially upon the correlations determined during the machine-learning stage, and based at least partially upon the one or more conditions that the given patient is suspected of having, determining a set of questions to ask the patient such that determining that the patient has one of the one or more conditions with a likelihood that passes a threshold likelihood may be achieved with a minimum number of questions being asked;
   based upon the determined set of questions, choosing a next question to ask the given patient; and outputting the next question to the given patient, upon an output device.

There is further provided, in accordance with some applications of the present invention, apparatus for performing a differential diagnosis of a patient, the apparatus including:

at least one output device; and

at least one computer processor configured:
in a machine-learning stage:
   to receive data relating to a plurality of patients, and to receive conditions that respective patients belonging to the plurality of patients are diagnosed as having; and
   to thereby generate predictive models that relate patient-related data to patient diagnoses; and in a patient-diagnosis stage:
   to receive a plurality of parameters relating to a given patient;
   in response to the received parameters, and using the predictive models that were determined during the machine-learning stage, to diagnose the patient as having one or more conditions; and
   in response thereto:
      to generate an output on the at least one output device indicating the one or more conditions that that the given patient has been diagnosed as having; and
      to generate an output on the at least one output device indicating a contribution of a given portion of the received parameters, toward diagnosing the patient as having the one or more conditions.

In some applications, the computer processor is configured to receive the plurality of parameters relating to the given patient at least partially by outputting questions to the patient, and receiving answers to the questions.

In some applications, the computer processor is configured to receive the plurality of parameters relating to the given patient at least partially by automatically receiving parameters relating to the given patient's medical history.

In some applications, the computer processor is configured to receive the plurality of parameters relating to the given patient at least partially by automatically measuring one or more physiological parameters of the given patient, using one or more sensors.

In some applications, the computer processor is configured to receive the plurality of parameters relating to the given patient at least partially by automatically acquiring one or more images of the given patient.

In some applications, the computer processor is configured to receive the plurality of parameters relating to the given patient at least partially by automatically measuring one or more physiological parameters of the given patient, using one or more robotic components.

In some applications, the computer processor is configured to generate the output indicating the contribution of the given portion of the received parameters toward diagnosing the patient as having the one or more conditions by indicating a weighting of a given one of the received parameters in diagnosing the patient as having the one or more conditions.

In some applications, the computer processor is configured to generate the output indicating the contribution of the given portion of the received parameters toward diagnosing the patient as having the one or more conditions by indicating respective weightings of a plurality of respective received parameters in diagnosing the patient as having the one or more conditions.

In some applications, the computer processor is configured to generate the output indicating the contribution of the given portion of the received parameters toward diagnosing the patient as having the one or more conditions by generating an output in which a correlation between a given set of the received parameters and the one or more conditions is indicated.

There is further provided, in accordance with some applications of the present invention, a method for performing a differential diagnosis of a patient, the method including:

using at least one computer processor:
in a machine-learning stage:
   receiving data relating to a plurality of patients, and receiving conditions that respective patients belonging to the plurality of patients are diagnosed as having; and
   thereby generating predictive models that relate patient-related data to patient diagnoses; and in a patient-diagnosis stage:
   receiving a plurality of parameters relating to a given patient;
   in response to the received parameters, and using the predictive models that were determined during the machine-learning stage, diagnosing the patient as having one or more conditions; and
   in response thereto:
      generating an output indicating the one or more conditions that that the given patient has been diagnosed as having; and
      generating an output indicating a contribution of a given portion of the received parameters, toward diagnosing the patient as having the one or more conditions.

There is additionally provided, in accordance with some applications of the present invention, a computer software product, for performing a differential diagnosis of a patient and for use with an output device, the computer software product comprising a non-transitory computer-readable medium in which program instructions are stored, which instructions, when read by a computer cause the computer to perform the steps of:

in a machine-learning stage:
receiving data relating to a plurality of patients, and receiving conditions that respective patients belonging to the plurality of patients are diagnosed as having; and thereby determining correlations between respective patient parameters and the conditions that patients are diagnosed as having; and in a patient-diagnosis stage:
determining one or more conditions that a given patient is suspected of having;
based at least partially upon the correlations determined during the machine-learning stage, and based at least partially upon the one or more conditions that the given patient is suspected of having, determining a set of questions to ask the patient such that determining that the patient has one of the one or more conditions with a likelihood that passes a threshold likelihood may be achieved with a minimum number of questions being asked;
based upon the determined set of questions, choosing a next question to ask the given patient; and
outputting the next question to the given patient, upon the output device.

There is additionally provided, in accordance with some applications of the present invention, a computer software product, for performing a differential diagnosis of a patient, the computer software product comprising a non-transitory computer-readable medium in which program instructions are stored, which instructions, when read by a computer cause the computer to perform the steps of:

in a machine-learning stage:
receiving data relating to a plurality of patients, and receiving conditions that respective patients belonging to the plurality of patients are diagnosed as having; and thereby generating predictive models that relate patient-related data to patient diagnoses; and

in a patient-diagnosis stage:
receiving a plurality of parameters relating to a given patient;
in response to the received parameters, and using the predictive models that were determined during the machine-learning stage, diagnosing the patient as having one or more conditions; and
in response thereto:
   generating an output indicating the one or more conditions that that the given patient has been diagnosed as having; and
   generating an output indicating a contribution of a given portion of the received parameters, toward diagnosing the patient as having the one or more conditions.

The present invention will be more fully understood from the following detailed description of applications thereof, taken together with the drawings, in which:

### BRIEF DESCRIPTION OF THE DRAWINGS

FIGS. 1A and 1B show two parts of a flowchart of a method for increasing the efficiency and accuracy of the passage of a patient through an emergency room procedure, in accordance with some applications of the present invention;

FIGS. 2A and 2B show two parts of a flowchart of a method of operating a patient-testing station, in accordance with some applications of the present invention;

FIG. 3 is a schematic illustration of a patient-testing station, which is a robotic station that incorporates an anthropomorphic robotic mechanism for performing palpable examination of a patient, in accordance with some applications of the present invention;

FIGS. 4A and 4B are schematic illustrations of patient-testing stations, which are robotic stations incorporating an automated electrocardiography (ECG) apparatus, in accordance with some applications of the present invention;

FIG. 5 is a schematic illustration of a robotic portion of a patient-testing station that performs automatic blood collection, in accordance with some applications of the present invention;

FIG. 6 is a flowchart showing steps of a method that are performed, in accordance with some applications of the present invention; and

FIG. 7 is a flowchart showing steps of a method that are performed, in accordance with some applications of the present invention.

### DETAILED DESCRIPTION OF EMBODIMENTS

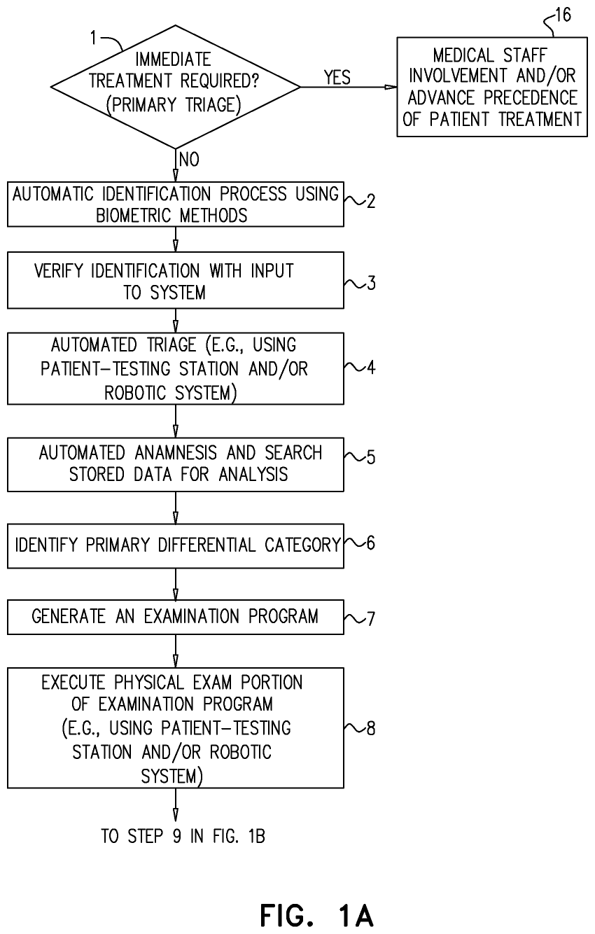

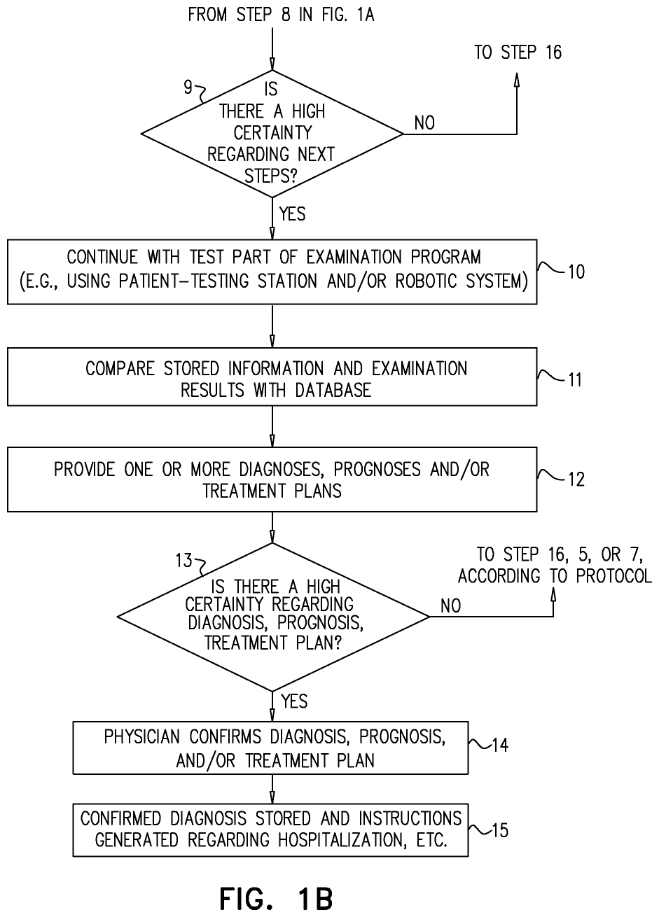

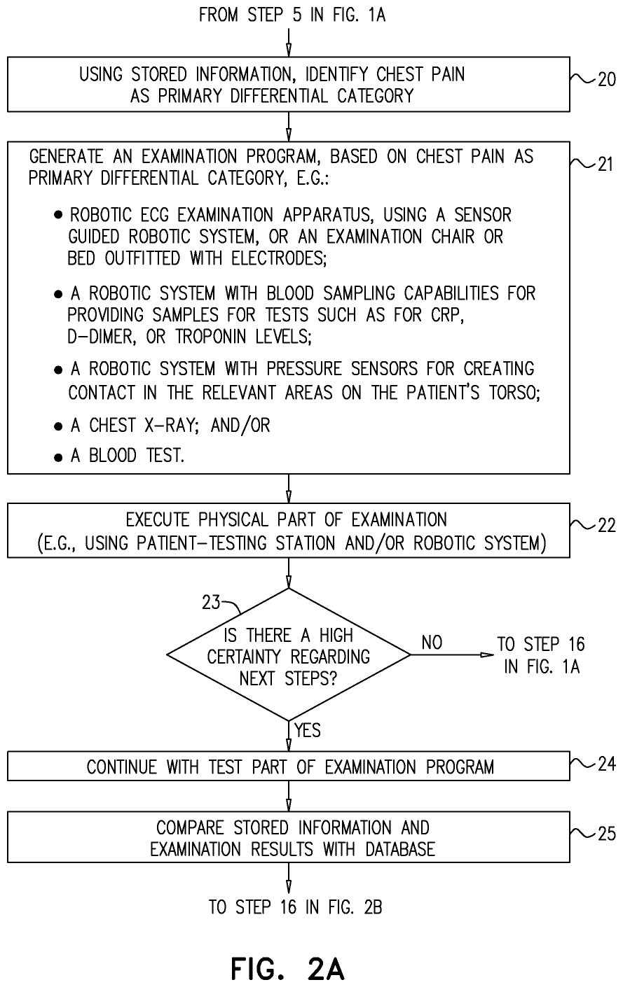

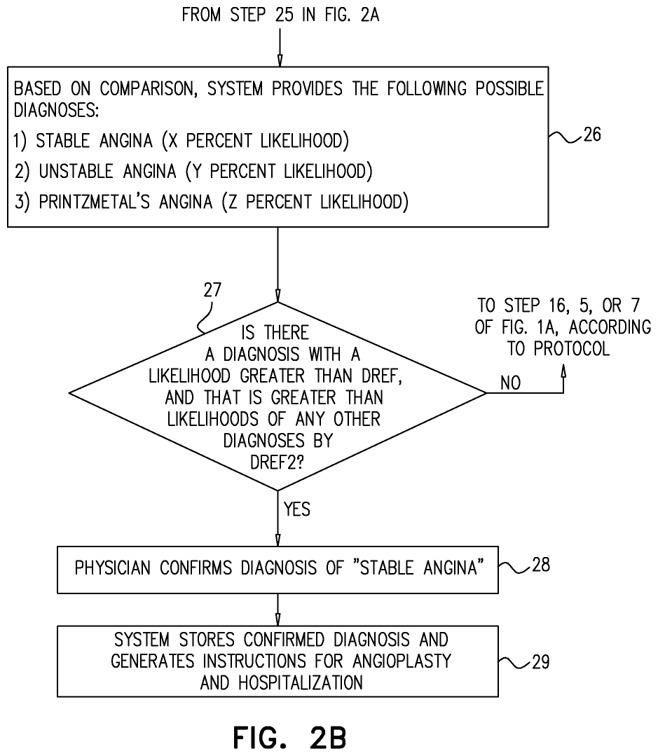

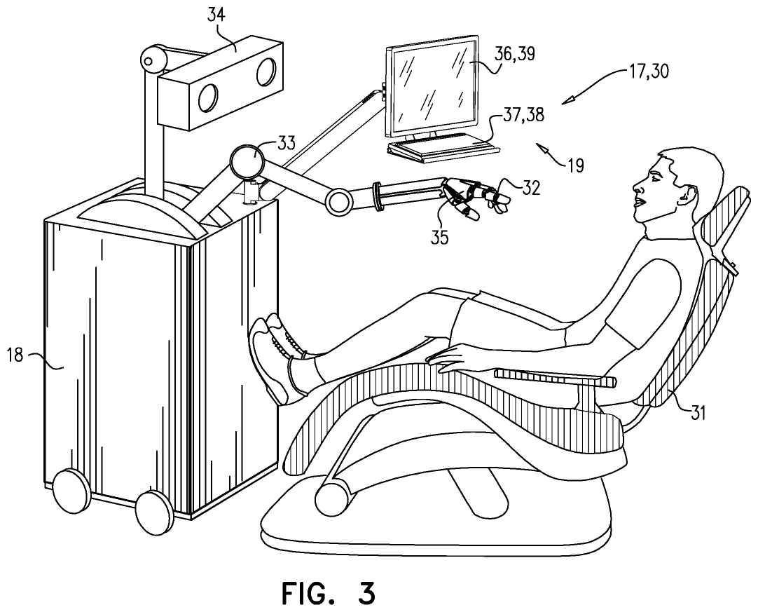

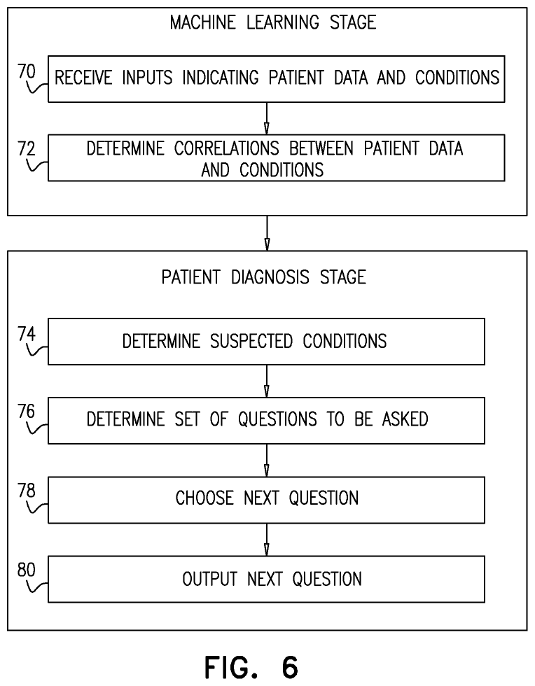

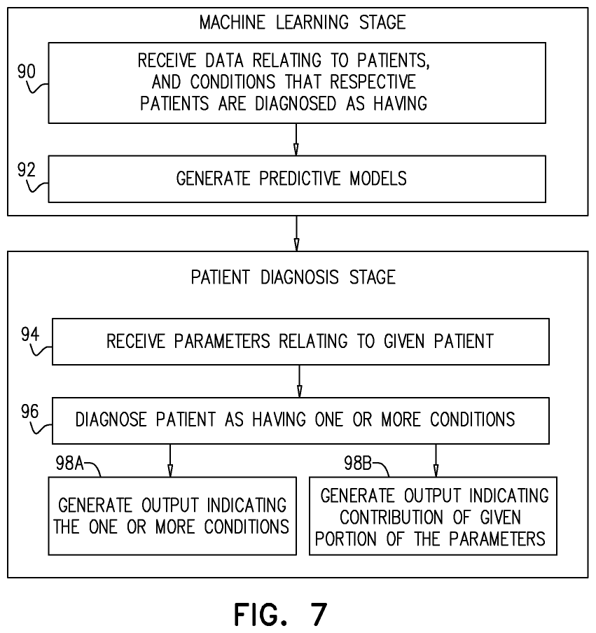

Reference is now made to FIGS. 1A and 1B, which show respective portions of a flow chart of a typical procedure used as a patient passes through the emergency room of the hospital, equipped with an intake and analysis system, in accordance with some applications of the present invention. Reference is also made to FIGS. 3-5, which show examples of a patient-testing station 17, or components thereof, in accordance with some applications of the present invention. Typically, procedures as described with reference to FIGS. 1A-B are performed using a patient-testing station. Although the examples of the patient-testing station shown in FIGS. 3-5 include certain robotic components, the scope of the present invention includes performing the procedures described with reference to FIGS. 1A-B, as well as those described with reference to FIGS. 2A-B and FIGS. 6-7, with a patient-testing station that does not include such robotic components, mutatis mutandis.

Referring to FIG. 3, typically, the patient-testing station includes a computer processor 18. For some applications, computer processor 18 is in-built to the patient-testing station, as shown. Typically, the computer processor communicates with a memory, and with a user interface 19. The patient, a person accompanying the patient, and/or a medical staff member typically sends instructions to the computer processor, via an input device 37 of the user interface. For some applications, the input device includes a keyboard 38 (as shown in FIG.

3, for example), a mouse, a joystick, a touchscreen device (such as a smartphone or a tablet computer), a touchpad, a trackball, a voice-command interface, and/or other types of input devices that are known in the art. Typically, the computer processor generates an output via an output device 36 of the user interface. For some applications, the output device includes a monitor 39 (as shown in FIG. 3, for example), and the output includes an output that is displayed on the display. For some applications, the computer processor generates an output on a different type of visual, text, graphics, tactile, audio, and/or video output device, e.g., speakers, headphones, a smartphone, or a tablet computer. For example, the computer processor may generate an output on an output device associated with a given healthcare professional, and/or a given set of healthcare professionals. For some applications, the processor generates an output on a computer-readable medium (e.g., a non-transitory computer-readable medium), such as a disk, or a portable USB drive, and/or generates an output on a printer.

Referring now to the procedure that is described in the flowchart of FIGS. 1A-1B, the procedure is typically performed using patient-testing station 17. For some applications, the patient-testing station is configured to triage, diagnose, and/or recommend treatment for a patient in an autonomous or semi-autonomous manner. It is noted that in the context of the present application, the term autonomous should be understood as referring to a process or system that does not require input from a healthcare professional (such as a doctor or a nurse). An autonomous process or system as described herein, typically does require patient compliance, and may additionally include certain preliminary steps to be performed by a healthcare professional, for example, in order to instruct the patient how to use the system and/or in order to perform a primary triage (e.g., as described hereinbelow, with reference to step 1 of FIG. 1A). The term semi-autonomous should be interpreted as referring to a process or system that generally proceeds without requiring input from a healthcare professional (such as a doctor or a nurse), but may occasionally use such input, for example, as described hereinbelow. It is further noted that steps that are described hereinbelow as being performed by the “system” are typically performed by one or more components of patient-testing station 17.

Typically, patient-testing station 17 is configured to output questions to the patient via output device 36, and to receive answers to the questions from the patient via input device 37.

For some applications, computer processor 18 of the patient-testing station is configured to receive data relating to the patient by accessing the patient's medical history records, as described in further detail hereinbelow. Typically, the computer processor obtains additional data relating to the patient by performing tests upon the patient, e.g., by performing such tests in an autonomous or semi-autonomous manner using robotic components, sensors, and/or imaging components of the patient-testing station, as described in further detail hereinbelow. The computer processor typically performs the methods described herein, and generates an output to the patient and/or to a healthcare professional, e.g., an emergency room doctor or nurse. For some applications, the computer processor communicates with one or more additional computer processors (not shown). For example, computer processors that are disposed remotely from computer processor 18 may store machine-learning data, and/or medical history records, and computer processor 18 may access such data by communicating with the one or more remotely-disposed computer processors.

In step 1 of the procedure shown in FIGS. 1A-B, the patient arrives at the emergency room, and an initial primary triage is performed by the receiving medical staff, to ascertain whether the patient requires immediate attention in the CCU (Coronary Care Unit), in the neurosurgical unit (e.g., if a serious stroke is suspected), and/or in the respiratory unit (e.g., if the patient's condition is deemed life-threatening). If so, the patient does not enter the usual emergency room routine, but is handled by a medical team because of the potential criticality of his/her condition. This is shown in step 16. At any point during the whole procedure, if a patient's situation becomes critical or life-threatening as is indicated by the system, the method may return to step 16 for medical staff involvement and/or advanced precedence of treatment. However, after step 16 has been employed, for example, if the patient's condition has stabilized, the medical staff may decide that there should be a return to using a generally autonomous system and the patient may be returned to any step in FIG. 1A or FIG. 1B that the staff deems appropriate. Typically, an autonomous system is or is not used depending on the specific medical assessment of the staff. A purely human, or combined human and automated (e.g., artificial-intelligence-based) collaborated procedure, or purely autonomous procedure is typically performed, according to the vital signs and symptoms aggregation, and possibly also based on the patient's illness history and medical records.

Typically, less critical patients are handled at step 2, in which the patient's identity is automatically checked using either a facial imaging device, a fingerprint or handprint device, an iris image signature, and/or any similar system for biometric identification. This identification is optionally backed up by the scanning of the patient's ID card or other similar identification document in step 3. If, however, a patient is not recognized by the system in step 2, or it is known that the patient will not be recognized, then step 2 may be omitted and step 3 may be used alone.

The patient's identity is typically used to register the patient by one of two paths; either the patient is identified in the hospital records, and then all that he/she has to do is to verify that identity in step 3, or, for a new patient, the patient or his accompanying person has to register with personal details in step 3.

In step 4, for patients not determined in the primary triage to be in a life-threating situation, an automatic triage procedure is performed, the implementation being based on a sensor guided robotic system, cameras, and/or sensors (e.g., non-contact, contact non-invasive, and/or contact minimal invasive sensors), and optionally being artificial intelligence based.

In step 5, upon reaching the patient's turn in the triage, the patient undergoes an interactive automated anamnesis session, and at the same time, the system searches accessible records for any relevant historical medical data on that particular patient. Such relevant and accessible historical medical data is understood to be included in the stored data of step 5, subsequent steps, and throughout this disclosure. If relevant historical medical data on the patient is not found, the system proceeds with only the information obtained during the automated anamnesis session.

In step 6, the results of the anamnesis session, and optionally of the historical medical data accessed in step 5, are used to identify a primary differential illness group or category, for example “chest pain,” upon which a tailored examination program will be based. Such an examination program may include a physical examination portion, which may include examinations based on sensor-guided robotic systems, and a testing portion which may include blood tests, urine tests, etc. In step 7, an examination program is generated based on the primary differential illness category, which defines the routine and the non-routine tests, images and consultations which are advised to be performed on that patient.

In step 8, the patient undergoes the physical tests and imaging prescribed by the examination program. Automatic physical examinations are typically performed using patient-testing station 17. For some applications, the patient-testing station is based on sensor-guided robotic systems, and/or remote manipulation by a doctor, who can give instructions to the robotic system to check a specific response of a patient, which the overseeing doctor believes to be necessary, and which is not in the examination protocol of the station itself. The test procedures can all be performed in one patient-testing station (which may, for example, have multiple robotic systems for executing various tests), or separate stations may be used for each specific test or group of tests, with the patient transferred between the separate stations. If at any point during this process, test results are generated which cannot be readily associated with one or more diagnoses, there may be protocol regarding the need to repeat tests for the purpose of yielding more applicable results.

In step 9, based on the results from the physical examination and other steps that have been performed, the system makes a decision as to whether there is a high enough certainty regarding the next steps to enable the system to continue autonomously. If so, in step 10, automatic standing orders are initiated for tests such as imaging, blood tests, urine tests, etc., based on the examination program. If not, then the system proceeds to step 16, in which an attending doctor is required to review the case, and the doctor decides whether or not to proceed with the standing order tests. The certainty of the system to proceed autonomously may be based on a comparison of the physical exam results to a database, for example, to determine the likelihood of providing a diagnosis based on such results, or to identify unusual results that may require further examination by a doctor. As mentioned previously, the system may determine that there should be additional tests in an attempt to gain results that will offer higher certainty, instead of referring directly to step 16.

In step 11, the totality of intake and historical data, and of all of the test results generated, is compared with a background database of patients whose symptom profiles and/or test results are similar to those of the patient being treated, and an assessment of the likely diagnoses, and, optionally, suggested treatments and/or prognoses, is performed and is output to the emergency room digital records system. At this stage, there may be one or more likely diagnoses provided. As described in further detail hereinbelow, typically this comparison is performed using machine-learning techniques.

In step 12, one or more diagnoses, prognoses, and/or treatment plans are provided, based upon the comparison performed in step 11. In step 13, the system, based on the data generated, decides if there is a high certainty regarding the final diagnosis, and optionally prognosis and treatment plan, and if so, it is sent for review by the attending physician in step 14, so that a confirmed diagnosis and optionally prognosis and treatment plan may be made. In situations in which there more than one likely diagnosis is indicated, a grading system is typically provided, based on the statistical likelihood of the accuracy of each of the possible diagnoses. Additionally, in a situation in which more than a predetermined number of diagnoses are provided by the system, additional tests or information regarding the patient's current condition may be accumulated to reduce the number of likely diagnoses, such as to reduce the diagnoses to below a predetermined number.

For some applications, if the final diagnosis and prognosis does not have a high certainty of accuracy, predetermined protocol is followed to determine whether the case is returned to the medical staff at step 16, for further review and testing, or is returned to step 5 or step 7 to obtain new information and/or new test results. For example, this may be obtained by performing new tests and/or asking new questions, and/or by repeating previous tests and/or questions. Alternatively or additionally, further historical data is accumulated in this step. Such a lack of high certainty may occur, for example, when there are many suggested diagnoses by the system that are all given a high statistical probability or grading. As another example, even if there are only two suggested diagnoses, but they both have a 50 percent probability of accuracy according to the grading system, then this may also constitute a lack of high certainty and may warrant further information or testing.

After a physician has confirmed a diagnosis in step 14, the system, in step 15, stores the confirmed diagnosis and generates instructions for the next steps regarding hospitalization or check out and follow up.

It is to be understood that the procedure shown in FIG. 1A and FIG. 1B is only one exemplary way in which the progress through an emergency room or other medical setting can be described, and although certain steps and their order are mandatory, in some applications of the invention, other steps are omitted, amended, or re-ordered, e.g., according to the procedure of the specific emergency room or medical clinic involved.

Reference is now made to FIGS. 2A and 2B, which show respective parts of a flowchart showing sub-steps of the method of FIG. 1A and 1B in which, by way of example, “chest pain” is determined as the primary differential illness category (e.g., based upon information gained during anamnesis and from historical medical records, in accordance with steps 1-5 of Figs. 1A and 1B), in accordance with some applications of the present invention. As described with reference to FIGS. 1A-B, typically the procedure described with reference to FIGS. 2A-2B are performed using patient-testing station 17. FIGS. 2A and 2B show a procedure that includes generation of an examination program and performing the tests required for a patient whose primary differential category has been determined to be “chest pain”. Typically, at least some of the steps described with reference to FIGS. 2A-2B are performed automatically by a computer processor, such as computer processor 18 of patient-testing station 17 (FIG. 3).

In step 20, the system uses the patient's data from the initial stages of accepting and generating the intake of the patient and accessible medical records, in accordance with steps 1-5 of FIGS. 1A-1B. Based on that data, the system identifies a primary differential category of “chest pain”. If, in this step, the system is unable to determine a primary differential category, the patient may be referred to medical staff as in step 16 of Figs. 1A and 1B.

On the basis of the primary differential category, a tailored examination program is determined for the patient, of which typical details are outlined, in step 21. The patient is sent, in step 22, to a patient-testing station (such as patient-testing station 17 described herein), e.g., an automatic physical examination station, which may include:

a robotic ECG/EKG examination apparatus, using a sensor guided robotic system, or an examination chair or bed outfitted with electrodes;

a robotic system with blood sampling capabilities for providing samples for tests such as for CRP, D-dimer, or Troponin levels;

a robotic system with pressure sensors for creating contact in the relevant areas on the patient's torso;

a chest x-ray; and/or

a blood test.

For some applications, the patient-testing station includes an analysis system for performing analysis of one or more of the above-mentioned tests, in situ.

Once the automated physical tests have been concluded, in step 23 the system decides if there is high certainty regarding the next steps that should be taken. If there is not high certainty, the patient's case may be referred to medical staff, in accordance with step 16 of FIGS. 1A and 1B, to possibly be returned to the autonomous procedure at a later stage. If there is high certainty, the procedure progresses through the testing portion of the examination program in step 24. High certainty may be determined using any statistical methods, such as identifying patterns that have a high correlation with previously obtained data that is related to a diagnosis, or identifying unusual test results that may warrant further testing. After the testing has been completed, the system then compares all stored information and examination results with a database in step 25.

Typically, in performing the comparison of step 25, the system uses artificial intelligence methods and detects correlations to previous clinical patterns to provide one or more likely diagnoses in step 26. If the system is unable to generate a likely diagnosis, the system may determine that further testing is warranted or may refer the patient to the medical staff in step 16 of FIGS. 1A and 1B. In the example shown, in step 26, three likely diagnoses are generated by the system, and each is graded according to its statistical probability of accuracy, using percentages, weighted numbers or any other relative quantitative methods. As an example, the system may output that the patient has an X percent likelihood of stable angina as an accurate diagnosis, Y percent likelihood of unstable angina as an accurate diagnosis, and a Z percent likelihood of Prinzmetal's angina as an accurate diagnosis. Typically, the system additionally generates one or more likely prognoses and treatment plans, at this stage, also based on the comparison of step 25.

Step 27 illustrates an exemplary algorithmic step in which the system determines if there is a high enough certainty regarding an accurate diagnosis from the diagnoses of step 26. This discrimination may be performed using any suitable statistical method. In the example shown, the system determines if there is a single diagnosis of the likely diagnoses generated in step 26, which has more than a predetermined level of likelihood of accuracy, Dref, and which exceeds the likelihood of accuracy of each of the other diagnoses of step 26 by at least a second predetermined value, Dref2. To continue the aforementioned example, if, for instance, Dref=80 percent, and, for instance, Dref2=10 percent, and if X percent is higher than 80 percent and has more than 10 percent likelihood of accuracy over Y percent and over Z percent, there is shown a single diagnosis which fulfills these requirements for providing a diagnosis having a high certainty of accuracy. Thus, in the example shown, the patient would be diagnosed as having “stable angina”, X percent being associated with the diagnosis of “stable angina”. The system may then also determine if there is a high accuracy regarding the prognosis and treatment plan at this stage.

Thus, in this example, there is determined to be a high certainty regarding the diagnosis. However, the hospital protocol may mandate that all automatic diagnoses be confirmed by a human physician. Under these circumstances, the method may then proceed to step 28 in which the physician may then confirm the diagnosis of “stable angina”. The physician may optionally confirm or determine of his/her own accord a prognosis and a treatment plan at this stage. In step 29, the system stores the confirmed diagnosis and generates instructions for hospitalization, or for check out and follow up. In the example shown, the system generates instructions for hospitalization and an angioplasty procedure.

Reference is now made to FIGS. 3 to 5, which schematically illustrate some examples of patient-testing station 17 and/or components thereof, the patient testing-station including a robotic station 30, in accordance with some applications of the present invention. For some applications, the patient-testing station uses sensors and testing routines in order to accomplish tasks such as the automatic testing and imaging of a patient. Typically, the apparatus and methods described with reference to FIGS. 3-5 are used in conjunction with the apparatus and methods described with reference to FIGS. 1A-B and 2A-B.

For some applications, robotic station 30 includes robot activation arm or arms. For some such applications, the arms have palpating facilities, imitating those activated by a human doctor, enabling the arms to make physical contact with the patient where necessary, in order to perform a bodily examination. In order to successfully achieve most of the functions of the stations described, the robotic station may be equipped with some form of artificial vision, with optical or other sensors on any robotically controlled arm to view and assure the position of the patient being examined. For some applications, the robotic station includes or works in conjunction with image-processing facilities in order to analyze and focus on the region which the robotic station is intended to interact with. For some applications, the robotic station communicates with the patient (e.g., via user interface 19, shown in FIG. 3), in order to perform interactive, physical contact with the patient.

For some applications, a robotic station is used to extract a sample of bodily fluids from the patient. For some such applications, the activation arm or arms are equipped with equipment for withdrawing the sample, e.g., equipment for drawing a saliva sample, or equipment (such as a syringe), in order to make a puncture in order to draw blood, as described in further detail hereinbelow.

For some applications, computer processor 18 is configured to perform artificial-intelligence processing. For some applications, the computer processor is configured to access databases that include records not only of standard expected situations and responses with regard to any bodily part being examined, but also a large bank of historic diagnostic responses to such examinations. In this way, by using such historical data banks, and by use of deep learning procedures, the results are more accurately interpreted. Further typically, the database is updated based upon tests that are performed on each patient, the corresponding diagnoses, and/or the accuracy of such diagnoses.

For some applications, robotic station 30 is a general-purpose station, and is used, for example, for palpable interactive examinations of the patient, the patient's temperature, the patient's sweating level, and force reaction examinations. For some applications, the robotic station is equipped with specific instruments, sensors or cameras, in order to fulfill specific tests or image-based examinations. Typically, the robotic station is adapted to perform multiple examinations, to increase efficiency and to save the need for movement of the patient from one station to another.

Referring again to FIG. 3, for some applications, robotic station 30 incorporates an anthropomorphic robotic mechanism (e.g., an arm) 33, for performing palpable examination of the patient. Such a station can be used for measuring the patient's physical reaction to force, such as when the doctor wishes to measure the strength of a limb, such as an arm, or a hand. For example, the doctor may remotely grab and manipulate the limb to feel the resistance, as is done in neurological exams, or the robotic mechanism may be used for palpable examination of the patient's anatomy, especially internal organs. The station may include a bed (not shown) for such tests as abdominal examinations, a seat 31, or a standing cubicle.

The robotic mechanism includes one or more artificial feeling extremities 35, typically shaped as a human hand (as shown). Typically, the extremities have flexibility and agility similar to a human hand. For some applications, the patient-testing station (e.g., the robotic station) includes one or more imaging devices 34 for imaging the patient. For some applications, the imaging device include a three-dimensional imaging system, and the computer processor is configured to relate the position of the feeling extremities with the region of the patient's body which is being examined, using three-dimensional images acquired by the imaging system. For some applications, the robotic mechanism incorporates robotically activated artificial fingers 32 that are configured to find and feel the organs being examined. For some such applications, the fingers have force feedback sensors and/or tactile sensors, which are configured to extract meaningful data regarding size, consistency, texture, location, and tenderness of the organ or body part being palpated. For example, such a robotic station can be used for abdominal examinations, for breast examinations, and/or for orthopedic examination of muscular or bone damage.

For some applications, the robotic station is equipped with user interface 19, which is typically generally as described hereinabove. During the examination, the robotic station may be programmed to ask the patient questions, e.g., relating to the level of pain during motion or during palpation, or during pressure, or relating to the limits of motion during manipulation of limbs, or similar questions of the type that a human doctor would ask patient during such a bodily contact examination.

For some applications, the robotic station is configured to operate at least partially autonomously, based either on artificial-intelligence algorithms, on a programed pre-determined routine, or both. For example, the robotic station may use sensor guiding of the robotic system with feedback from the patient. For some applications, in addition to operating in an autonomous mode, the station is also configured to be operated by a remotely-located physician, who can operate or provide guidelines to the robotic system to check a specific response of the patient. For some applications, an interactive remote terminal that provides haptic feedback is provided to assist the doctor in this task.

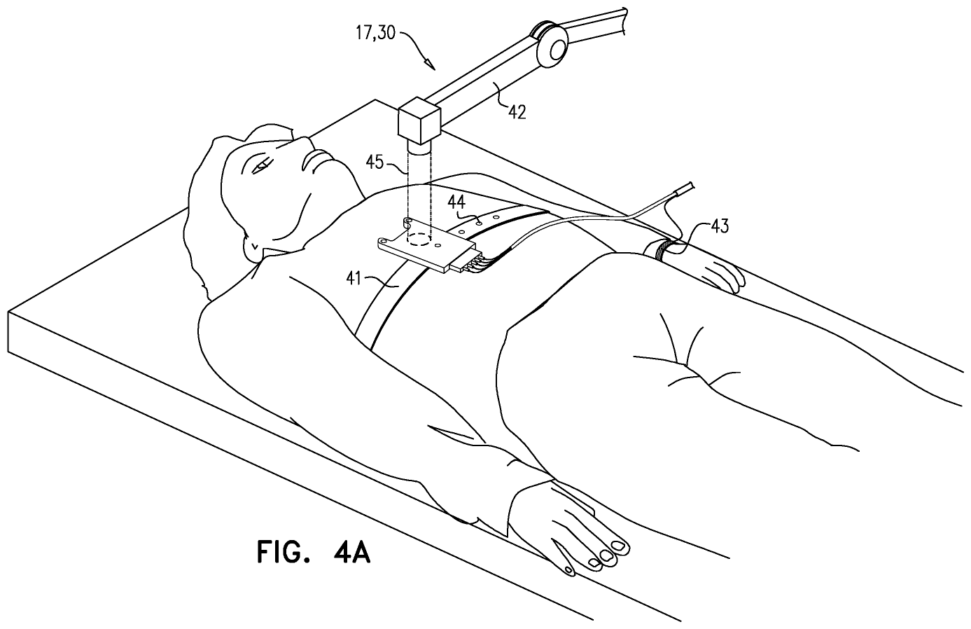

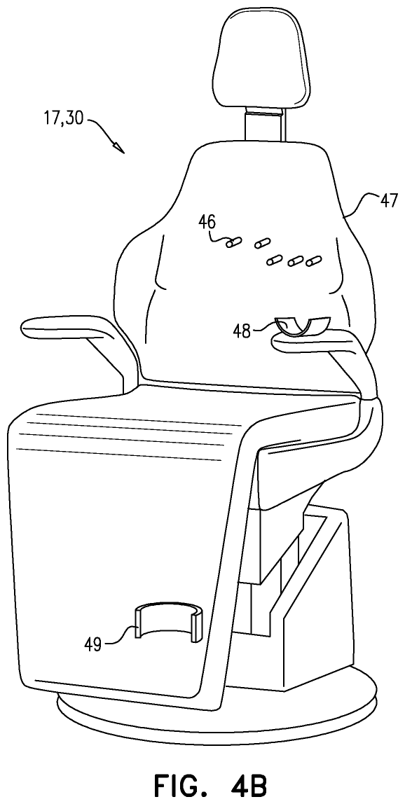

Reference is now made to FIGS. 4A and 4B, which are schematic illustration of robotic components of patient-testing station 17, the patient-testing station being configured to perform an automated electrocardiography (ECG) test, in accordance with some applications of the present invention. In this manner, the patient-testing station is configured to record the electrical activity of the heart over a period of time using electrodes placed on the patient skin or over his/her clothing. For some applications, the patient-testing station is configured to conduct a full 12-lead ECG, in which electrodes are placed on the surface of the chest and on the patient's limbs, in order to determine the clinical status of the patient.

As shown in FIG. 4A, for some applications an ECG electrode set 44 is attached to a semi-rigid curved arm 41. For some applications, curved arm 41 is placed on the patient's chest, using a programmed robotic arm 42, which is typically sensor guided to ensure correct placement of the electrodes on the patient's chest. Alternatively or additionally, a gantry robotic arm is used for the placement of the curved arm on the patient's chest.

For some applications, semi-rigid curved arm 41 includes a sternum plate of a semi-rigid ECG electrode belt, such as is provided by LevMed Ltd., of Zichron Ya'akov, Israel. The position of the electrode belt upon the patient's chest is typically maintained by its weight, or by positive robotic arm pressure. For some applications, the patient-testing station also connects electrode clamps 43 to the peripheral limb extremities, e.g., the patient's wrists and/or ankles. For some applications, electrical contact between the electrodes and the patient's skin is facilitated by robotic application of electrode solution to the skin-contact side of the electrodes, such as by a fluid pump and fine channels or pipes. For some applications, once there is electrical contact between the electrodes and the patient's skin, the robotic arm withdraws a connection 45 used to position the ECG belt, and the robotic station activates the ECG recording until a useful trace is obtained. This can be ascertained either by analysis by the computer processor of the trace output, or by remote inspection by the attending doctor. For some applications, the robotic station thus enables the entire ECG procedure to be performed without active participation of attending medical personnel, and typically within a relatively short space of time (e.g., less than 5 minutes, e.g., approximately 2 minutes), depending on the cooperation of the patient.

Although the normal ECG procedure is for the patient to undergo the test after at least partially undressing, such that the electrodes touch the skin directly, for some applications, the patient-testing station is configured to perform ECG testing on a clothed patient, e.g., using drops of electrode contact solution applied automatically to the clothing which the tips of the ECG electrodes touch, to maintain electrical contact between the skin surface and the tips of the ECG electrodes. For some applications, the patient-testing station utilizes a closed loop feedback system to continue dispensing solution until the ECG signal at any electrode is sufficiently identifiable. For some applications, such an arrangement saves time in performing the ECG test, relative to if the patient were required to become undressed.

Reference is now made to FIG. 4B, which is a schematic illustration of patient-testing station, the patient-testing station including robotic components configured to measure an ECG of a patient, in accordance with some applications of the present invention. In the application shown in FIG. 4B, the electrodes are applied to the back and/or sides of the patient, by means of spring-loaded electrodes 46 positioned in the appropriate locations in the back of an examination chair 47 (the back of the chair typically being curved), or the surface of an examination bed (not shown in FIG. 4B). In this manner, the ECG can be performed posteriorly on the back of the patient, for example, using the V7, V8 and V9 placement positions, or any other suitable protocol positions. The position of the electrodes must be carefully aligned with the patient's back in order to ensure that the electrodes are accurately positioned opposite the tissue the electrical impulses of which they are intended to detect. For some applications, the chair includes a vertically movable seat configured to facilitate the correct positioning of the electrodes, or a motion mechanism configured to move the electrode set, both of these options typically being controlled by an automatic patient position sensor (e.g. using a three-dimensional imaging system, as described hereinabove). As in the anterior station shown in FIG. 4A, the posterior ECG can also be performed on a clothed patient. For some applications, a wrist-band electrode 48 and/or an ankle band electrode 49 are also provided for the limb leads.

In an alternative application, not shown in the drawings, electrodes are used that project from a bed on which the patient is instructed to lie, preferably in the prone position to provide optimum signal from the cardiac region, but also possibly in the supine position for a posterior ECG examination.

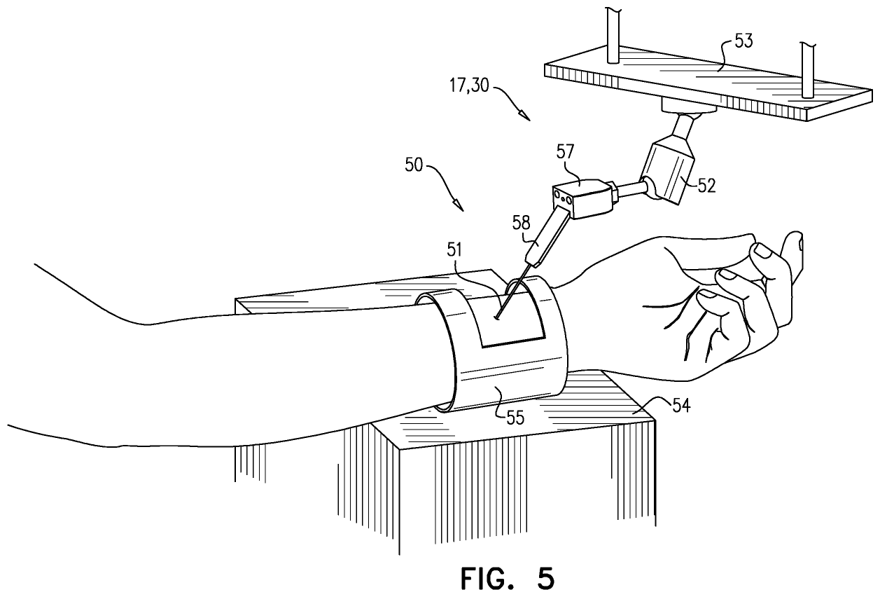

Reference is now made to FIG. 5, which is a schematic illustration of robotic components of patient-testing station 17, the robotic components being configured to perform blood collection by venipuncture, in accordance with some applications of the present invention. For some applications, the patient-testing station is configured to obtain vascular access by insertion of a needle 51 or catheter into the patient's vein, using a fully or partially automatic system. For some applications, as shown in FIG. 5, a robotic manipulator 52 is supported on a gantry 53, located above an arm-rest 54 incorporating a cuff 55, used to constrain the patient's arm 56 while the automatic blood extraction is performed. The needle is typically accurately inserted into the vein of the patient, using automatic guidance, e.g., using one of several methods described hereinbelow. For some applications, an ultrasound probe applied to the skin of the patient's arm is used in order to investigate the vascular structure or blood flow through Doppler sensing beneath the skin in the region of the venipuncture. Typically, an ultrasound image processing system is applied to the ultrasound images to determine the position of the intended vein, relative to the cuff and arm-rest. The position of the arm rest is predefined relative to the station gantry and hence the robotic coordinates. The patient-testing station thus accurately relates the needle position to the intended position as determined from the acquired and analyzed images. Alternatively, the patient-testing station may use a near infrared imaging system 57, as shown in FIG. 5, to generate a 3-dimensional map of the subcutaneous blood vessels, and image processing of this map may be used by the computer processor to provide input instructions to robotic manipulator 52. Typically, the robotic manipulator aligns needle drive mechanism module 58 for insertion of the needle once the position has been determined.

For some applications, an infra-red vein imager is used, such as the VeinViewer® Vision product, provided by Christie Medical Products of Memphis, Tenn., which uses near-infrared light to project a digital image of the patient vasculature directly onto the surface of the skin. A conventional video image processor is used to guide the robotic needle inserter to the correct angle and location, based upon the digital image of the patient vasculature on the surface of the skin. Alternatively, direct image processing of the image is used to guide the robotically aligned needle. For some applications, a bio-sensitive detection system is used in order to ensure that the needle or catheter has been inserted into a blood vessel and not into surrounding tissue. Typically, once the needle location within the vein has been confirmed, a mechanical mechanism, preferably within needle drive mechanism module 58, is used to connect a vacuum container or a flexible collection tube to the needle end to draw the blood. Use of such an automated venous puncture station typically enables the speed and accuracy of blood collection to be substantially increased over a manual system operated by medical personnel.

For some applications, blood testing is performed using alternative or additional methods, for example, using a drop of capillary blood obtained from a finger prick, and applying micro fluid detection techniques on that drop of blood. Alternatively or additionally, a patch of micro-needles, typically measuring only a few millimeters (e.g., up to 20 mm) across, is applied to the skin of the patient, to withdraw bodily fluid, which may be analyzed within the micro-needles themselves. For some applications, the needles are at least 1 mm in length, so that they penetrate to the epidermis and the dermis, such that it is possible to draw and analyze capillary blood from a small area. For some such applications, each micro-needle, or group of micro-needles, is adapted to execute a blood test for a different blood component, depending on the reagent contained in the micro-needle, and the method used for the analysis. For some applications, spectral analysis is used to analyze the blood in the micro-needles. Alternatively or additionally, other analysis methods are used. For some applications, use of such a patch analysis method enables the patient-testing station, or the portion thereof that is used for blood analysis, to perform blood analysis without the need for any significant accuracy of placement of the patch upon the patient's skin.

The above-described patient-testing stations, robotic stations, robotic components, and techniques for use therewith, are examples of the type of examinations which can be performed using the emergency room autonomous, semi-autonomous, and/or robotic techniques of the present disclosure. Additional examples include the use of infrared imaging (e.g., using imaging device 34 of FIG. 3) to perform other diagnostic functions. One such example is an automated examination station, in which far infrared imaging of the patient's facial features is used in order to determine a number of clinical parameters. The sensitivity of the thermal imaging region to changes in body temperature can be used in order to determine such features as noncontact pulse rate, the patient's pulse being clearly visible in a high definition thermal image of the patient's face. Other uses of such thermal imaging techniques include the detection of growths in soft tissue, enlarged organs such as the thyroid, peripheral blood vessel abnormalities, detection of trauma to the head, secondary brain injury, brain bleeding, intra-cranial hematoma, stroke, muscular and skeletal injuries, and the like.

For some applications, a combination of the tests that are described herein as being performed by patient-testing station 17 and/or robotic station 30 are performed on a single patient, e.g., using a single patient-testing station, or using a combination of patient-testing stations each of which has different testing capabilities.

For some applications, a computer processor as described herein is configured to combine multiple sources of medical information regarding the patient's status, as obtained, inter alia, from:

(i) tests and examinations performed using a patient-testing station, a robotic station, and/or robotic components, e.g., using non-contact, contact and minimally invasive sensors,

(ii) machine interpretation and processing of medical images generated during the tests,

(iii) information obtained automatically and interactively at a patient-testing station, and

(iv) the patient's medical history,

together with artificial intelligence interrogation of large databanks of medical situations for comparing with and analyzing the above assembled patient medical information.

Typically the computer processor connects to historical databases of previous measurements (e.g., textual, structural, and/or visual measurements, etc.), and uses machine-learning based methods and/or general artificial-intelligence methods to mine patterns with high correlation to previously given patient anamnesis, diagnosis, prognosis, etc. Using the derived patterns, the computer processor typically applies such patterns to the analysis of current patients to classify them based on the historical pattern to the most probable diagnosis, prognosis etc.

Typically, the computer processor thus enables the speedy and accurate assessment of the patient's clinical condition within a medical setting, whether that assessment is uniquely determined, or whether it results from a choice of more than one potential condition, combined with recommendations regarding the preferred continued treatment of the patient. In situations where there is indicated more than one likely diagnosis, a grading system is typically provided, based on the statistical likelihood of the accuracy of each of the possible diagnoses, e.g., as described hereinabove. Additionally, in a situation in which more than a predetermined number of diagnoses are provided by the system, additional tests or information regarding the patient's current condition may be accumulated to reduce the number of likely diagnoses, such as to reduce the diagnoses to below a predetermined number, e.g., in accordance with the techniques described herein. For some applications, a summary of all cases is referred to an attending doctor, in order to confirm the feasibility and logic of the determined diagnosis, especially in cases in which the system indicates that more than one diagnosis is possible, and a decision has to be made regarding which course of treatment to follow, e.g., in accordance with the techniques described hereinabove.

Reference is now made to FIG. 6, which is a flowchart showing steps of a method that are performed, in accordance with some applications of the present invention. As described hereinabove, for some applications, computer processor 18 of patient-testing station 17 utilizes artificial-intelligence methods to mine patterns with high correlation to previously given patient anamnesis, diagnosis, prognosis, etc. Using the derived patterns, the computer processor typically applies such patterns to the analysis of current patients to classify them based on the historical patterns to the most probable diagnosis, prognosis etc.

A machine-learning stage is typically performed at least partially by at least one computer processor that is remote from computer processor 18 of patient-testing station 17. During the machine-learning stage, the at least one computer processor receives data relating to a plurality of patients, as well as conditions that respective patients belonging to the plurality of patients are diagnosed as having (step 70 of FIG. 6). By analyzing the aforementioned inputs, the at least one computer processor determines correlations between respective patient parameters and the conditions that the patients are diagnosed as having (step 72 of FIG. 6). By way of example, the computer processor may determine that whether or not a patient feels chest pains is highly correlated with diagnosing a patient as suffering from a cardiac arrest, or it may determine that the age and/or sex of a patient is highly correlated with their susceptibility to liver disease.

During a patient diagnosis stage, a given patient is typically assessed, for example using the apparatus and methods described hereinabove. For some applications, in step 74, at least one computer processor (which typically includes computer processor 18 of patient-testing station 17) determines that the patient is suspected as having one or more conditions. For some applications, step 74 corresponds with step 6 of FIG. 1A, step 12 of FIG. 1B, and/or step 26 of FIG. 2B. For example, the computer processor may determine the one or more conditions that the patient is suspected of having, by asking the patient a preliminary set of questions, by automatically measuring one or more physiological parameters of the given patient using one or more sensors and/or one or more imaging devices, by automatically measuring one or more physiological parameters of the given patient using one or more robotic components, and/or by accessing the patient's medical history.

Subsequently, in step 75, based at least partially upon the correlations determined during the machine-learning step, and based at least partially upon the one or more conditions that the given patient is suspected of having, the computer processor determines a set of questions to ask the patient. Typically, the set of questions is determined by selecting a set of questions that is such as to (a) determine that the patient has one of the one or more conditions with a likelihood that passes a threshold likelihood, with (b) a minimum number of questions being asked.

Purely by way of example, the threshold likelihood may be 90 percent. It may be determined that based at least partially upon the correlations determined during the machine-learning step, and based at least partially upon the one or more conditions that the given patient is suspected of having, there is a set of five questions, based upon which (depending upon the answers that the patient gives), it may be possible to diagnose the patient as suffering from one of the one or more conditions with more than a 90 percent likelihood. It may further be determined that there is a different set of six questions, based upon which (depending upon the answers that the patient gives), it may be possible to diagnose the patient as suffering from one of the one or more conditions with more than a 90 percent likelihood. Therefore, the computer processor may determine that the set of five questions should be asked. For some applications, in determining the set of questions that the patient should be asked, the computer processor uses natural language processing to determine which words to use in the questions.

For some applications, additional considerations are taken into account when determining the set of questions to ask the subject. For example, the computer processor may be able to diagnose the patient as suffering from one of the one or more conditions with more than the threshold likelihood either by (a) asking the patient a first set of questions, or by (b) performing tests on the subject in combination with asking the subject a second set of questions that is shorter than the first set of questions. In such a case, the computer processor may choose to ask the first set of questions, even though it is longer, because this may minimize the number of questions that need to be asked, without additionally requiring performance of the tests. Thus, more generally, for some applications, when determining the set of questions to ask the subject, the computer processor accounts for additional parameters (e.g., test results, imaging results, and/or medical history parameters) regarding the patient that may be required in order to diagnose the patient as suffering from one of the one or more conditions with more than the threshold likelihood. The computer processor determines for a given set of additional parameters that may be required, a set of questions that could be combined with the additional parameters that would be such as to (a) determine that the patient has one of the one or more conditions with a likelihood that passes a threshold likelihood, with (b) a minimum number of questions being asked.

For some applications, the method then proceeds to step 78, in which the computer processor chooses which question to ask the patient next, based upon the output of step 76. To continue with the example provided in the above paragraph, of the set five questions that were determined in step 76, the computer processor may choose the question the answer to which would carry the greatest weight in diagnosing the patient as suffering from one of the one or more conditions with more than a 90 percent likelihood.

For some applications, during the machine-learning stage, the computer processor identifies a question that can be asked to a patient in order to resolve a contradiction between responses that the patient has given to two or more previous questions. Subsequently, in the patient-diagnosis stage, the computer processor chooses to ask the patient the identified question, in response to the given patient having given responses to two or more previous questions that result in the contradiction.

Typically, the steps described with reference to the patient diagnosis stage of FIG. 6 are performed in a continuous manner. For example, if the patient's response to the next question is such that it becomes more likely that the patient is suffering from a different condition, or such that a new set of questions could be asked that would allow the computer processor to arrive at a diagnosis having a likelihood that exceeds the threshold using fewer questions, then a new set of questions may be chosen, and a new next question may be chosen. For some applications, based upon the answers that the patient gives, the threshold likelihood for the diagnosis is adjusted. To continue with the example given above, if the computer processor determines that based upon the responses that have been received, the maximum possible likelihood for a correct diagnosis is only 80 percent, then the computer processor may apply the steps described hereinabove using the lower threshold.

It is noted that, typically, the machine-learning stage is ongoing and temporally overlaps with the patient-diagnosis stage, inasmuch that at the same time as a given patient is diagnosed, data relating to that patient is fed into the one or more computer processors that are configured to perform the machine-learning analysis of the data. It is further noted that the data received in step 70 of the machine-learning stage may be received automatically, e.g., by being received from the computer processors of patient-testing stations, and/or by being received from other sources, e.g., by analyzing the medical records of a large number of patients that are stored on a database.

Reference is now made to FIG. 7, which is a flowchart showing steps of a method that are performed, in accordance with some applications of the present invention. As described hereinabove with reference to FIG. 6, for some applications, a procedure is performed in which there is a machine-learning stage (which is typically performed at least partially by one or more computer processors that are remote from patient-testing station) and a patient-diagnosis stage (which is typically performed at least partially by computer processor 18 of patient-testing station 17). Also as described hereinabove with reference to FIG. 6, for some applications, the machine-learning stage is ongoing and temporally overlaps with the patient-diagnosis stage.

For some applications, in step 90, at least one computer processor receives data relating to a plurality of patients, and receives conditions that respective patients belonging to the plurality of patients are diagnosed as having. As described with reference to step 70 of FIG. 6, the data received in step 90 of the machine-learning stage may be received automatically, e.g., by being received from the computer processors of patient-testing stations, and/or by being received from other sources, e.g., by analyzing the medical records of a large number of patients that are stored on a database. In step 92, the computer processor generates predictive models that relate patient-related data to patient diagnoses, for example, using machine-learning techniques.

During the patient-diagnosis phase, in step 94, at least one computer processor (e.g., computer processor 18 of patient-testing station 17) receives parameters relating to a given patient. For example, such parameters may be obtained using techniques as described hereinabove, e.g., by asking the patient questions via user interface 19, by automatically measuring one or more physiological parameters of the given patient using one or more sensors and/or imaging devices, by automatically measuring one or more physiological parameters of the given patient using one or more robotic components, and/or by accessing the patient's medical history.

In step 96, in response to the received parameters, and using the predictive models that were determined during the machine-learning step, the computer processor diagnoses the patient as having one or more conditions. In step 98A, the computer processor generates an output indicating the one or more conditions that the patient is suspected of having, and in step 98B (which is typically performed concurrently with step 98A, and using the same output device as that used for step 98A), the computer processor generates an output indicating the contribution of a given portion of the parameters, toward diagnosing the patient as having the one or more conditions. For some applications, the outputs described with reference to steps 98A and 98B are generated on user interface 19 of the patient-testing station. Alternatively or additionally, the outputs are sent to a healthcare professional, such as an attending doctor or nurse. For example, a printout may be generated for the healthcare professional, or the outputs may be generated on a device that is used by the healthcare professional.

By way of example, in step 98B, the computer processor may generate an output indicating that the main contributing factor toward diagnosing the patient as having a given condition was his/her answer to a given question, was the result of a given test that was performed, was the result of an image that was acquired, was the result of an item in his/her medical history, etc. Alternatively or additionally, the computer processor may indicate a weighting of a given one of the parameters (or respective weightings of a plurality of parameters) in diagnosing the patient as having the given condition. For some applications, the computer processor generates a textual explanation of how the diagnosis was arrived at. For example, the text may include a description of a correlation between a given set of the received parameters and a condition that the patient has been diagnosed as having.

Typically, by outputting the indication of the contribution of a given portion of the parameters toward diagnosing the patient as having the one or more conditions, as described with reference to step 98B, the confidence of the patient, and moreover, the confidence of the doctor in the machine-generated diagnosis is strengthened. Alternatively, by outputting the indication of the contribution of a given portion of the parameters toward diagnosing the patient as having the one or more conditions, the doctor is able to better assess whether he/she agrees with the diagnosis, and/or whether he/she would like any additional tests or examinations to be performed.

It is noted that for some applications, the machine-learning and artificial intelligence related techniques described herein (e.g., the techniques described with reference to FIGS. 6 and 7) are performed by a patient-testing station which does not perform some or any of the functions described hereinabove with reference to FIGS. 3-5. For example, patient-testing station may include computer processor 18 and user interface 19, and may be configured to diagnose a patient using machine-learning techniques, based upon receiving responses of the patient to questions, based upon access to the patient's medical records, and/or based upon tests and or imaging that do not necessarily rely upon robotic components.

Applications of the invention described herein can take the form of a computer program product accessible from a computer-usable or computer-readable medium (e.g., a non-transitory computer-readable medium) providing program code for use by or in connection with a computer or any instruction execution system, such as computer processor 18. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-RAY) and DVD.

A data processing system suitable for storing and/or executing program code will include at least one processor (e.g., computer processor 18) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that blocks of the flowcharts shown in the figures and combinations of blocks in the flowcharts, can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processor 18) or other programmable data processing apparatus, create means for implementing the functions/acts specified in the flowcharts and/or algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the flowchart blocks and algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the flowcharts and/or algorithms described in the present application.

Computer processor 18 is typically a hardware device programmed with computer program instructions to produce a special purpose computer. For example, when programmed to perform the algorithms described with reference to the figures, computer processor 18 typically acts as a special purpose patient-analysis computer processor. Typically, the operations described herein that are performed by computer processor 18 transform the physical state of a memory, which is a real physical article, to have a different magnetic polarity, electrical charge, or the like depending on the technology of the memory that is used.

For some applications, operations that are described as being performed by a computer processor are performed by a plurality of computer processors in combination with each other.

There is therefore provided, in accordance with some applications of the present invention, the following inventive concepts:
Inventive concept 1. A method for managing the treatment of a patient in a medical setting, using an autonomous system, said method comprising:

assigning an identity to said patient using at least one of (i) biometric identification and (ii) patient input to said autonomous system;

for a patient determined to be in non-life-threatening condition, performing a procedure to determine precedence of the treatment of said patient relative to other patients;

accumulating information relevant to the current condition of the patient; performing a series of tests to ascertain current clinical parameters of said patient, at least some of said tests being indicated by the results of previous tests or by said accumulated information;

using said autonomous system to combine said accumulated information and said current clinical parameters to generate a combination parameter set;

using said autonomous system to compare said combination parameter set with a database to find previously obtained clinical patterns having high correlation to said combination parameter set; and

using the results of said comparison to determine one or more likely diagnoses for the clinical condition of said patient.
Inventive concept 2. The method according to inventive concept 1, wherein said step of determining one or more likely diagnoses for the clinical condition of said patient comprises the use of at least one of machine-learning based methods and Artificial Intelligence.
Inventive concept 3. The method according to inventive concept 1 or inventive concept 2, further comprising determining at least one of (i) at least one prognosis for said patient, and (ii) at least one proposal for the continued treatment of said patient.
Inventive concept 4. The method according to any one of inventive concepts 1-3, wherein said accumulated information further comprises information relating to the medical history of said patient.
Inventive concept 5. The method according to inventive concept 1, wherein determining each of said one or more likely diagnoses for the clinical condition of said patient comprises determining each of said one or more likely diagnoses for the clinical condition of said patient with a likelihood of accuracy.
Inventive concept 6. The method according to inventive concept 5, further comprising determining a single diagnosis for said patient from said one or more likely diagnoses if (i) a likelihood of accuracy of said single diagnosis is greater than a first predetermined value and (ii) said likelihood of accuracy of said single diagnosis is greater than likelihoods of accuracy of each of the other likely diagnoses by more than a second predetermined value.
Inventive concept 7. The method according to any one of inventive concepts 1-6 wherein, if said comparison indicates more than a predetermined number of likely diagnoses, said method further comprises:

performing additional tests to acquire additional clinical parameters relating to said patient to generate an enhanced combination parameter set;

comparing said enhanced combination parameter set with a database to find previously obtained clinical patterns having high correlation to said enhanced combination parameter set; and

using clinical patterns having high correlation to said enhanced combination parameter set in order to reduce the number of likely diagnoses for the clinical condition of the patient.
Inventive concept 8. The method according to inventive concept 7, wherein said step of performing additional tests further comprises accumulating additional information relating to said patient.
Inventive concept 9. The method according to inventive concept 7 or inventive concept 8, further comprising using said comparison of said enhanced combination parameter set to said database to provide at least one of (i) at least one prognosis for said patient, and (ii) at least one proposal for the continued treatment of said patient.
Inventive concept 10. The method according to inventive concept 8, wherein said accumulated additional information further comprises information relating to the medical history of said patient.
Inventive concept 11. The method according to any one of inventive concepts 1-10, wherein any of said databases include medical parameters from a large group of patients.
Inventive concept 12. The method according to inventive concept 1, wherein performing said procedure to determine precedence of the treatment of said patient comprises performing said procedure at least partially using a robotic system that includes at least one of (i) cameras and (ii) sensors, to acquire clinical data relating to the current condition of said patient.
Inventive concept 13. The method according to inventive concept 1 or inventive concept 12, wherein performing said procedure to determine precedence of the treatment of said patient comprises using artificial intelligence to analyze said clinical data.
Inventive concept 14. The method according to inventive concept 7, wherein performing the additional tests comprises determining which additional tests to perform, based on the results of previous tests, at least some of which were performed by a robotic system.
Inventive concept 15. The method according to inventive concept 14, wherein determining which additional tests to perform comprises determining which additional tests to perform, based on a high likelihood of results of said additional tests to do at least one of: (i) reduce the number of likely diagnoses and (ii) determine a single most likely diagnosis.
Inventive concept 16. The method according to any one of inventive concepts 1-15, wherein said method enables an increase in the accuracy of the diagnosis of the patient's illness, as compared to a diagnosis generated without use of an autonomous system.
Inventive concept 17. The method according to any one of inventive concepts 1-16, wherein said method enables a reduction in the time taken to process a patient through said treatment, as compared to a method not using an autonomous system.
Inventive concept 18. The method according to any one of inventive concepts 1-17, wherein performing the series of tests comprises performing at least one test that is a palpable examination of said patient, performed by an automated station.
Inventive concept 19. The method according to inventive concept 18, wherein performing the palpable examination of said patient comprises using a robotic hand to provide information relating to the patient's reaction to an applied force.
Inventive concept 20. The method according to inventive concept 19, wherein using said robotic hand comprises controlling the robotic hand remotely, or manipulating said robotic hand by means of a controller using a predetermined protocol in co-ordination with sensors for overseeing said manipulation.
Inventive concept 21. The method according to any one of inventive concepts 1-20, wherein performing the series of tests comprises performing at least one test that is an ECG examination of said patient, performed by an automated station.
Inventive concept 22. The method according to inventive concept 21, wherein performing said ECG examination comprises performing said ECG examination using a robotically placed set of electrodes mounted on a semi-rigid electrode arm.
Inventive concept 23. The method according to inventive concept 22, wherein performing said ECG examination comprises providing conductive fluid for said electrodes from a dispensing system of said semi-rigid electrode arm.
Inventive concept 24. The method according to inventive concept 21, wherein performing said ECG examination comprises performing said ECG examination using a bed for disposing said patient thereupon, including a set of electrodes protruding from the surface of said bed.
Inventive concept 25. The method according to inventive concept 21, wherein performing said ECG examination comprises performing said ECG examination using an examination chair for said patient, said chair including a set of electrodes protruding from its back.
Inventive concept 26. The method according to any of inventive concepts 22, 23, and 25, wherein performing said ECG examination comprises performing said ECG examination using electrodes that are either in predetermined positions, or are adjustable according to either electronic or visual feedback relating to the position of said patient.
Inventive concept 27. The method according to inventive concept 24 or inventive concept 25, wherein performing said ECG examination comprises performing said ECG examination using said set of protruding electrodes the electrodes including a dispensing system for providing conductive fluid for said electrodes.
Inventive concept 28. The method according to inventive concept 1, wherein performing the series of tests comprises obtaining a blood sample from the patient, using a robotic system.
Inventive concept 29. The method according to inventive concept 28, wherein obtaining the blood sample from the patient comprises obtaining the blood sample from the patient, by venipuncture.
Inventive concept 30. The method according to inventive concept 28, wherein obtaining the blood sample from the patient comprises obtaining the blood samples from the patient, using a micro-needle patch, and performing analysis of the blood sample using said micro-needle patch.
Inventive concept 31. The method according to inventive concept 28, wherein obtaining the blood sample from the patient comprises obtaining the blood samples from the patient by a needle prick.
Inventive concept 32. The method according to any one of inventive concepts 1-31, wherein performing the series of tests comprises using a robotic cranial scanning device for ascertaining a presence of a stroke.
Inventive concept 33. The method according to any one of inventive concepts 1-32, wherein accumulating information relevant to the current condition of the patient comprises performing an automated anamnesis procedure that uses responses relating to the patient in order to generate subsequent questions, using the automated system.
Inventive concept 34. The method according to any one of inventive concepts 1-33, further comprising assigning priority in at least one of treatment and medical imaging based on said accumulated information relating to the current condition of said patient.
Inventive concept 35. The method according to any one of inventive concepts 1-34, wherein said medical setting is a hospital emergency room.
Inventive concept 36. The method according to any one of inventive concepts 1-35, wherein performing the series of tests comprises performing a series of tests, at least some of which are performed using a robotic system.
Inventive concept 37. The method according to any one of inventive concepts 1-36, wherein performing the series of tests comprises performing a series of tests, at least some of which are performed using a robotic system that includes an automated station adapted to perform more than one of said series of tests.
Inventive concept 38. A robotic test station for performing an ECG examination on a patient, comprising: a location adapted to receive said patient, said location comprising a surface adapted for contact proximate the position of patient's heart with a portion selected from the group consisting of: skin of the patient, and clothing over the patient's skin, wherein said surface comprises a set of electrodes protruding therefrom, said electrodes being disposed such that they detect electrical signals from the heart of said patient.
Inventive concept 39. The robotic test station according to inventive concept 38, further comprising a dispensing system for providing conductive fluid at said electrodes.
Inventive concept 40. The robotic test station according to inventive concept 39, wherein said dispensing system is adapted to continue dispensing solution until the ECG signal at any electrode is sufficiently identifiable.
Inventive concept 41. The robotic test station according to any one of inventive concepts 38 to 40, wherein said electrodes are in predetermined positions.

Inventive concept 42. The robotic test station according to any one of inventive concepts 38 to 40, wherein said electrodes are adjustable according to feedback relating to the position of said patient in said location, the feedback being selected from the group consisting of: electronic feedback and visual feedback.
Inventive concept 43. The robotic test station according to any one of inventive concepts 38 to 42, wherein said surface comprises a surface selected from the group consisting of: a back of an examination chair, and a surface of a bed.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-39. (canceled)

40. A method for managing the treatment of a patient in a medical setting, the method comprising:
using an autonomous patient-testing station that includes a robotic system:
assigning an identity to said patient using at least one of (i) biometric identification and (ii) patient input to said autonomous system;
accumulating data related to a current clinical condition of the patient;
using the robotic system, performing one or more physical tests to determine current clinical parameters of the patient;
determining one or more likely diagnoses for the current clinical condition of the patient, by analyzing data using a machine-learning classifier, the data including the accumulated data related to the current clinical condition of the patient and the current clinical parameters of the patient determined using the robotic system; and
generating an output indicating the one or more likely diagnoses.

41. The method according to claim 40, wherein performing the one or more physical tests comprises performing a palpable examination of the patient, using the robotic system.

42. The method according to claim 41, wherein performing the palpable examination of the patient using the robotic system comprises using a robotic hand to provide information relating to the patient's reaction to an applied force.

43. The method according to claim 42, wherein using the robotic hand comprises controlling the robotic hand remotely.

44. The method according to claim 42, wherein using the robotic hand comprises manipulating the robotic hand by means of a controller using a predetermined protocol in coordination with sensors for overseeing said manipulation

45. The method according to claim 40, wherein performing the one or more physical tests using the robotic system comprises performing an ECG examination of the patient using the robotic system.

46. The method according to claim 45, wherein performing the ECG examination using the robotic system comprises performing the ECG examination using electrodes of the robotic system and providing conductive fluid for the electrodes from a dispensing system of the robotic system.

47. The method according to claim 45, wherein performing the ECG examination using the robotic system comprises performing the ECG examination using a robotically-placed set of electrodes mounted on a semi-rigid electrode arm.

48. The method according to claim 45, wherein performing the ECG examination using the robotic system comprises performing the ECG examination using a bed that includes a surface having a set of electrodes protruding therefrom.

49. The method according to claim 45, wherein performing the ECG examination using the robotic system comprises performing the ECG examination using an examination chair that includes a back having a set of electrodes protruding therefrom.

50. The method according to claim 40, wherein performing the one or more physical tests using the robotic system comprises obtaining a blood sample from the patient using the robotic system.

51. The method according to claim 50, wherein obtaining the blood sample from the patient using the robotic system comprises obtaining the blood sample from the patient, by venipuncture.

52. The method according to claim 50, wherein obtaining the blood sample from the patient using the robotic system comprises obtaining the blood sample from the patient, using a robotically-applied micro-needle patch.

53. The method according to claim 50, wherein obtaining the blood sample from the patient using the robotic system comprises obtaining the blood sample from the patient using a robotically-applied needle prick.

54. The method according to claim 40, wherein performing the one or more physical tests using the robotic system comprises using a robotic cranial scanning device for ascertaining a presence of a stroke.
